(19) United States
(12) Patent Application Publication
(10) Pub. No.: US 2009/0253616 A1
(43) Pub. Date: Oct. 8, 2009

(54) COMBINATION THERAPY FOR THE TREATMENT OF CANCER

(75) Inventor: Nicholas John Keen, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/300,865

(22) PCT Filed: May 14, 2007
(86) PCT No.: PCT/GB07/01754

§ 371 Date: Nov. 14, 2008

(30) Foreign Application Priority Data
May 16, 2006 (GB) 0609619.2

(51) Int. Cl. A61K 38/13; A61K 31/517; A61P 35/00

(52) U.S. Cl. 514/11; 514/266.23

(57) ABSTRACT

A combination comprising an aurora kinase inhibitor and an efflux transporter inhibitor wherein the aurora kinase inhibitor is a compound of formula (I) or pharmaceutically acceptable salt thereof for use in the treatment of hyperproliferative diseases such as cancer.

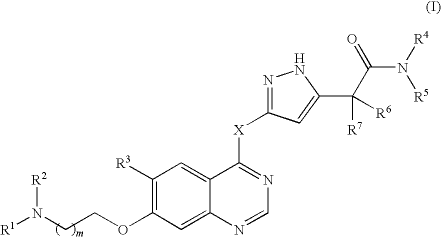

The present invention relates to a combination comprising an aurora kinase inhibitor and an efflux transporter inhibitor. The combination is useful in a new method of treating hyperproliferative diseases such as cancer. The invention also relates to a kit and a pharmaceutical composition comprising said combination and to the use of said combination in the manufacture of a medicament for use in the treatment of hyperproliferative diseases such as cancer.

Cancer (along with other hyperproliferative diseases) is characterised by uncontrolled cellular proliferation which occurs when normal regulation of cell proliferation is lost. This loss often appears to be the result of genetic damage to the cellular pathways that control the progression of a cell through its cell cycle.

In eukaryotes, an ordered cascade of protein phosphorylation is thought to control the cell cycle. Several families of protein kinases that play critical roles in this cascade have been identified. The activity of many of these kinases is increased in human tumours when compared to normal tissue. This can occur by either increased levels of expression of the protein (for example as a result of gene amplification), or by changes in expression of co activators or inhibitory proteins.

The first identified, and most widely studied of these cell cycle regulators have been the cyclin dependent kinases (or CDKs). More recently, protein kinases that are structurally distinct from the CDK family have been identified which play critical roles in regulating the cell cycle and which also appear to be important in oncogenesis. These include human homologues of the Drosophila aurora and S. cerevisiae Ip11 proteins. The three human homologues of these genes aurora-A, aurora-B and aurora-C (also known as aurora 2, aurora 1 and aurora 3 respectively) encode cell cycle regulated serine-threonine protein kinases (summarised in Adams et al., Trends in Cell Biology, 2001, 11(2): 49-54). These show a peak of expression and kinase activity through G2 and mitosis. Several observations implicate the involvement of human aurora proteins in cancer.

The aurora-A gene maps to chromosome 20q13, a region that is frequently amplified in human tumours including both breast and colon tumours. Aurora-A may be the major target gene of this amplicon, since aurora-A DNA is amplified and mRNA overexpressed in greater than 50% of primary human colorectal cancers. In these tumours aurora-A protein levels appear greatly elevated compared to adjacent normal tissue. In addition, transfection of rodent fibroblasts with human aurora-A leads to transformation, conferring the ability to grow in soft agar and form tumours in nude mice (Bischoff et al., The EMBO Journal, 1998, 17(11), 3052-3065). Other work (Zhou et al., Nature Genetics, 1998, 20(2), 189-93) has shown that artificial overexpression of aurora-A leads to an increase in centrosome number and an increase in aneuploidy, a known event in the development of cancer.

It has also been shown that there is an increase in expression of aurora-B (Adams et al., Chromsoma, 2001, 110(2), 65-74) and aurora-C (Kimura et al., Journal of Biological Chemistry, 1999, 274(11): 7334-40) in tumour cells when compared to normal cells.

Aurora-B is overexpressed in cancer cells and increased levels of aurora-B have been shown to correlate with advanced stages of colorectal cancer (Katayama et al., J. Natl Cancer Inst., 1999, 91, 1160). Furthermore, it has been reported that overexpression of aurora-B induces aneuploidy through increased phosphorylation of histone H3 at serine 10 and that cells overexpressing aurora-B form more aggressive tumours that develop metastases (Ota T. et al., Cancer Res., 2002, 62, 5168-5177). Aurora-B is a chromosome passenger protein which exists in a stable complex with at least three other passenger proteins, Survivin, INCENP and Borealin (Carnena M et al., Nat. Rev. Mol. Cell. Biol., 2003, 4, 842-854). Survivin is also upregulated in cancer and contains a BIR (Baculovirus Inhibitor of apoptosis protein (IAP) Repeat) domain and may therefore play a role in protecting tumour cells from apoptosis and/or mitotic catastrophe.

With regard to aurora-C, its expression is thought to be restricted to the testis although it has been found to be overexpressed in various cancer lines (Katayama H et al., Cancer and Metastasis Reviews, 2003, 22: 451-464).

Importantly, it has also been demonstrated that abrogation of aurora-A expression and function by antisense oligonucleotide treatment of human tumour cell lines (WO 97/22702 and WO 99/37788) leads to cell cycle arrest and exerts an antiproliferative effect in these tumour cell lines. Additionally, small molecule inhibitors of aurora-A and aurora-B have been demonstrated to have an antiproliferative effect in human tumour cells (Keen et al. 2001, Poster #2455, American Association of Cancer Research annual meeting), as has selective abrogation of aurora-B expression alone by siRNA treatment (Ditchfield et al., J. Cell Biology, 2003, 161(2), 267-280). This indicates that inhibition of the function of aurora-A and/or aurora-B will have an antiproliferative effect that may be useful in the treatment of human tumours and other hyperproliferative disease.

The inhibition of aurora kinases as a therapeutic approach to these diseases may have significant advantages over targeting signalling pathways upstream of the cell cycle (e.g. those activated by growth factor receptor tyrosine kinases such as epidermal growth factor receptor (EGFR) or other receptors). Since the cell cycle is ultimately downstream of all of these diverse signalling events, cell cycle directed therapies such as inhibition of aurora kinases would be predicted to be active across all proliferating tumour cells, whilst approaches directed at specific signalling molecules (e.g. EGFR) would be predicted to be active only in the subset of tumour cells which express those receptors. It is also believed that significant “cross talk” exists between these signalling pathways meaning that inhibition of one component may be compensated for by another.

Several small molecule inhibitors of aurora-A and/or aurora-B kinase for use in the treatment of hyperproliferative diseases such as cancer are already known, for example from WO03/55491 and WO2004/058781, (the contents of which are incorporated herein by reference). However the intracellular effectiveness of such inhibitors is dependent upon the extent to which they are retained by and/or distributed within hyperproliferating cells. The activity of an inhibitor in a cell may be significantly reduced if that inhibitor is a substrate of a cellular efflux transporter expressed by that cell. Efflux transporters protect cells from a wide variety of chemically unrelated toxins and drugs, which can cross the cell membrane by passive diffusion in the absence of transporters. Their expression has been linked to multi drug resistance (MDR) wherein a reduction in drug activity is observed when the efflux transporter decreases the total intracellular retention of the drug or redistributes the intracellular accumulation of the drug away from the site of action (Tan et al., Current Opinion in Oncology, 2000, 12: 450-458).

P-glycoprotein was the first efflux transporter to be identified as having a role in MDR (Chen et al., Cell, 1986, 47(3), 381; Seeling et al., Mini-review in Medicinal Chemistry, 2005, 135-151). Other efflux transporters known to have a role in cancer include BCRP, MRP1 and MRP2 (Fischer et al., Mini-Reviews in Medicinal Chemistry, 2005, 5, 183-195).

BCRP (also known as ABCG2, MXR, ABCP) is a 655 amino acid member of the ABCG family of ATP-binding cassette membrane transporters (Yanase K et al., Functional SNPs of the Breast Cancer Resistance Protein; Therapeutic Effects and Inhibitor Development, Cancer Lett., 2005), which is widely expressed in normal cells and tissues, such as capillary endothelial cells, haematopoietic stem cells, intestine, liver, breast, the maternal-fetal barrier of the placenta and the blood brain barrier (Fischer V. et al., Efflux Transporters and their Clinical Relevance, Mini-Reviews in Medicinal Chemistry, 5.2, 2005, 183-95; and Loscher W. et al., Role of Drug Efflux Transporters in the Brain for Drug Disposition and Treatment of Brain Diseases, Prog. Neurobiol, 76.1, 2005, 22-76). BCRP is a half transporter which functions as a dimer and it is believed that its functional role is to protect against the actions of toxic substances and metabolites in the tissues where it is expressed (Sugimoto Y. et al., Breast Cancer Resistance Protein: Molecular Target for Anticancer Drug Resistance and Pharmacokinetics/Pharmacodynamics, Cancer Sci., 96.8, 2005, 457-65). BCRP has been shown to confer multi-drug resistance in cells to a number of compounds including (but not limited to) topotecan, mitoxantrone, doxorubicin and SN-38 by ATP-dependent drug efflux (Doyle L A. et al., Multidrug Resistance mediated by the Breast Cancer Resistance Protein BCRP (ABCG2), Oncogene, 22.47, 2003, 7340-58; and Fischer et al.). Overexpression in certain tumour types may be associated with poor prognosis or resistance to treatment (Yoh K. et al., Breast Cancer Resistance Protein impacts Clinical Outcome in Platinum-based Chemotherapy for Advanced Non-Small Cell Lung Cancer, Clin Cancer Res, 10.5, 2004, 1691-97).

P-glycoprotein (also known as Pgp, ABCB1 and MDR1) is a transmembrane ATP dependent efflux transporter which removes substrates from cells in a unidirectional manner (Krishna et al., Current Medicinal Chemistry—Anti-Cancer Agents, 2001, 1, 163-174). Pgp is known to be overexpressed in number of haematological and solid tumours such as acute leukaemias, breast cancer, ovarian cancer, head and neck cancer, non-Hodgkin lymphoma and Kaposi sarcoma (Tan et al.) and it is known to contribute to multidrug resistance (MDR) (Seelig et al.). Like BCRP, Pgp has a variety of substrates, which vary widely in chemical structure and mechanism of action, such as anthracyclines (e.g. doxorubicin, daunorubicin and epirubicin), vinca alkaloids (e.g. vinorelbine, vincristine and vinblastine), epipodophyllotoxins (e.g. etoposide and teniposide), taxanes (e.g. paclitaxel and docetaxel), topotecan, dactinomycin and mitomycin C (Thomas et al., Cancer Control, 2003 10, 2, 159-165).

The breadth of substrate structure and mechanism make it very difficult to predict whether or not a particular substance will be a substrate of efflux transporter such as BCRP or Pgp. Indeed, breadth of substrate is a feature common to efflux transporter in general with substate ranging from small molecule pharmaceuticals and toxins to sugars, amino acids, glycans, chlosterol, phospholipids, peptides and proteins (Fischer et al.).

WO03/55491 and WO2004/058781 do not address the issue of efflux transporter liability and in particular BCPR and/or Pgp liability. They therefore do not disclose whether or not the inhibitors therein are substrates of BCRP, Pgp or any other efflux transporters.

We have discovered that a number of these inhibitors are in fact efflux transporter substrates and consequently have reduced activity in resistant cells. Fortunately, we have also discovered that their intracellular activity can be enhanced by administering the inhibitor in combination with an inhibitor of efflux transporter activity such as an inhibitor or BCRP activity and/or Pgp activity.

Accordingly, there is provided a combination comprising an aurora kinase inhibitor and an efflux transporter inhibitor wherein the aurora kinase inhibitor is a compound of formula (I) or pharmaceutically acceptable salt thereof:

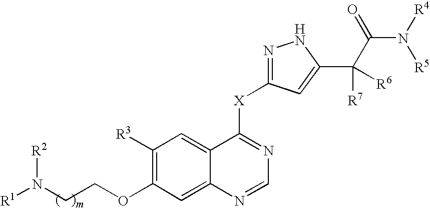

wherein:
m is 0, 1, 2 or 3
R¹ is hydroxyC₁₋₄alkyl or phosphonooxyC₁₋₄alkyl;
R² is hydrogen, C₁₋₄alkyl, hydroxyC₁₋₄alkyl, C₁₋₄alkoxyC₁₋₄alkyl or heterocyclyl;
or R¹ and R² together with the nitrogen to which they are attached form a 4- to 6-membered heterocyclic ring optionally containing a further heteroatom selected from nitrogen, oxygen and sulphur which nitrogen or sulphur is optionally oxidised and which ring is optionally substituted with C₁₋₄alkyl;
R³ is hydrogen or C₁₋₄alkoxy;
R⁴ is hydrogen or C₁₋₄alkyl;
R⁵ is aryl optionally substituted by 1 or 2 halo; and
R⁶ and R⁷ are independently hydrogen or C₁₋₄alkyl.

Within the present invention, it is to be understood that, insofar as certain compounds of formula (I) or a pharmaceutically acceptable salt thereof may exist in optically active or racemic forms by virtue of one or more asymmetric carbon or sulphur atoms, the invention includes in its definition any such optically active or racemic form which possesses aurora kinase inhibitory activity and in particular aurora A and/or aurora B kinase inhibitory activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to herein.

Within the present invention it is to be understood that a compound of formula (I) or a pharmaceutically acceptable salt thereof may exhibit the phenomenon of tautomerism and that the formulae drawings within this specification can represent only one of the possible tautomeric forms. It is to be understood that the invention encompasses any tautomeric form which has aurora kinase inhibitory activity and in particular aurora A and/or aurora B kinase inhibitory activity and is not to be limited merely to any one tautomeric form utilised within the formulae drawings.

It is also to be understood that certain compounds of formula (I) and pharmaceutically acceptable salts thereof can exist in solvated as well as unsolvated forms such as, for example, hydrated forms. It is to be understood that the invention encompasses all such solvated forms which have aurora kinase inhibitory activity and in particular aurora-A and/or aurora-B kinase inhibitory activity.

The present invention relates to the compounds of formula (I) as well as to pharmaceutically acceptable salts thereof. Pharmaceutically acceptable salts of the invention may, for example, include acid addition salts of compounds of formula (I) as herein defined which are sufficiently basic to form such salts. Such acid addition salts include but are not limited to furmarate, methanesulphonate, hydrochloride, hydrobromide, citrate and maleate salts and salts formed with phosphoric and sulphuric acid. In addition where compounds of formula (I) are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

The compounds of formula (I) may also be provided as in vivo hydrolysable esters. An in vivo hydrolysable ester of a compound of formula (I) containing carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

In this specification the generic term “alkyl” includes both straight-chain and branched-chain alkyl groups. However references to individual alkyl groups such as “propyl” are specific for the straight chain version only and references to individual branched-chain alkyl groups such as “tert-butyl” are specific for the branched chain version only.

“Heterocyclyl” is a saturated, unsaturated or partially saturated, monocyclic or bicyclic ring containing 4 to 12 atoms of which 1, 2, 3 or 4 ring atoms are chosen from nitrogen, sulphur or oxygen, which ring may be carbon or nitrogen linked, wherein a —CH₂— group can optionally be replaced by a —C(O)—; wherein a ring nitrogen or sulphur atom is optionally oxidised to form the N-oxide or S-oxide(s); and wherein a ring is optionally substituted by one or more halo or C₁₋₄alkyl. Examples include piperidinyl, piperazinyl, morpholinyl, tetrahydro-2H-pyranyl, pyrrolidinyl, pyrazolidinyl and imidazolidinyl.

“Phosphonooxy” is in one aspect a group of formula —OP(O)(OH)₂. However the term “phosphonooxy” also includes salts of this group such as those formed with alkali metal ions such as sodium or potassium ions or alkaline earth metal ions, for example calcium or magnesium ions.

Where optional substituents are chosen from “1 or 2” groups or substituents it is to be understood that this definition includes all substituents being chosen from one of the specified groups i.e. all substitutents being the same or the substituents being chosen from two or more of the specified groups i.e. the substitutents not being the same.

Compounds of the present invention have been named with the aid of computer software (ACD/Name version 8.0).

In one aspect of the invention, the aurora kinase inhibitor is a compound of formula (I) or pharmaceutically acceptable salt thereof wherein:

m is 1 or 2;
R¹ is hydroxyC₁₋₄alkyl or phosphonooxyC₁₋₄alkyl;
R² is C₁₋₄alkyl, C₁₋₄alkoxyC₁₋₄alkyl or a 6-membered saturated heterocyclyl;
or R¹ and R² together with the nitrogen to which they are attached form 6-membered heterocyclic ring optionally containing a further heteroatom selected from nitrogen, oxygen and sulphur which ring is optionally substituted with C₁₋₄alkyl;
R³ is hydrogen;
R⁴ is hydrogen;
R⁵ is phenyl optionally substituted by 1 or 2 halo; and
R⁶ and R⁷ are both hydrogen.

In another aspect the aurora kinase inhibitor is 2-{3-[(7-{3-[ethyl(2-hydroxyethyl)amino]propoxy}quinazolin-4-yl)amino]-1H-pyrazol-5-yl}-N-(3-fluorophenyl)acetamide, 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof.

In yet another aspect the aurora kinase inhibitor is 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof.

In one aspect of the invention the efflux transporter inhibitor is an inhibitor of the activity of BCRP. In another aspect of the invention the efflux transporter inhibitor is an inhibitor of the activity of Pgp.

In particular the efflux transporter inhibitor may be selected from resperpine, elacridar, fumitremorgin C (FTC), FTC analogues (such as KO0143), BIB-E, flavopiridol, CI1033 (quinazoline), gefitinib (Iressa), novobiocin, biricodar (VX-710), VX-853, diethylstilboestrol (DES), estrone, antioestrogens, tamoxifen and derivatives such as TAG-11, TAG-139, toremifine, imatinib mesylate, CI1033, estradiol, estriol, naringenin, acacetin, kaempferol and naringenin-7-glucoside. Further information regarding these efflux transporter inhibitors, which are believed to inhibit BCRP activity, can be found in Doyle L A. and Ross D D., Multidrug Resistance mediated by the Breast Cancer Resistance Protein BCRP (ABCG2), Oncogene 22.47, 2003, 7340-58 and Yanase K. et al., Functional SNPs of the Breast Cancer Resistance Protein; Therapeutic Effects and Inhibitor Development, Cancer Lett., 2005.

The efflux transporter inhibitor may also be selected from chlorpromazine, cyclosporin A, diltiazem, nicardipine, progesterone, quinidine, trifluoperazine, verapamil, BIBW22BS, dexniguldipine, gallopamil, PSC833, Roll-2933, trimethoxybenzoylyohimbine, biricodar (VX-710), elacridar (GF120918), zosuquidar (LY335979), MS-209, OC-144-093, laniquidar (R101933), S9788, tariquidar (XR9576), XR9051 and ONT-093. Further information regarding these efflux inhibitors, which are believed to inhibit Pgp activity, can be found in A. Seeling and E. Gatlik-Landwojtowicz, Mini-reviews in Medicinal Chemistry, 2005, 5, 135-151. In particular, the efflux transporter inhibitor may be selected from elacridar (GF120918), zosuquidar (LY335979), MS-209, OC-144-093, laniquidar (R101933), S9788, tariquidar (XR9576) and XR9051. The efflux transporter inhibitor may also be selected from BIBW22BS, dexniguldipine, gallopamil, PSC833, Roll-2933, trimethoxybenzoylyohimbine and VX-710. Alternatively, the efflux transporter inhibitor may be selected from chlorpromazine, cyclosporin A, diltiazem, nicardipine, progesterone, quinidine, trifluoperazine and verapamil.

In a particular aspect of the invention, the efflux transporter is elacridar.

The invention also provides a combination comprising an aurora kinase inhibitor and a compound selected from mitoxantrone, topotecan, irinotecan, SN-38, doxorubicin, daunorubicin, epirubicin, idarubicinol, flavopiridol, CI1033, BBR3390 and methotrexate, wherein the aurora kinase inhibitor is a compound of formula (I) as defined herein. In a particular embodiment of this aspect of the invention, the aurora kinase inhibitor is 2-{3-[(7-{3-[ethyl(2-hydroxyethyl)amino]propoxy} quinazolin-4-yl)amino]-1H-pyrazol-5-yl}-N-(3-fluorophenyl)acetamide, 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof, and more particluarly 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof.

A particular combination of the invention comprises 2-{3-[(7-{3-[ethyl(2-hydroxyethyl)amino]propoxy} quinazolin-4-yl)amino]-1H-pyrazol-5-yl}-N-(3-fluorophenyl)acetamide, 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof and an efflux transporter inhibitor selected from resperpine, elacridar, fumitremorgin C (FTC), FTC analogues (such as KO143), BIB-E, flavopiridol, CI1033 (quinazoline), gefitinib (Iressa), novobiocin, biricodar (VX-710), VX-853, diethylstilboestrol (DES), estrone, antioestrogens, tamoxifen and derivatives such as TAG-11, TAG-139, toremifine, imatinib mesylate, CI1033, estradiol, estriol, naringenin, acacetin, kaempferol and naringenin-7-glucoside. In a particular embodiment of this aspect of the invention, the aurora kinase inhibitor is 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof.

Another particular combination of the invention comprises 2-{3-[(7-{3-[ethyl(2-hydroxyethyl)amino]propoxy} quinazolin-4-yl)amino]-1H-pyrazol-5-yl}-N-(3-fluorophenyl)acetamide, 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof and a efflux transporter inhibitor selected from chlorpromazine, cyclosporin A, diltiazem, nicardipine, progesterone, quinidine, trifluoperazine, verapamil, BIBW22BS, dexniguldipine, gallopamil, PSC833, Roll-2933, trimethoxybenzoylyohimbine, biricodar (VX-710), elacridar (GF120918), zosuquidar (LY335979), MS-209, OC-144-093, laniquidar (R101933), S9788, tariquidar (XR9576), XR9051 and ONT-093. In a particular embodiment of this aspect of the invention, the aurora kinase inhibitor is 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof.

The aurora kinase inhibitors of the invention can be prepared by the methods described in WO03/55491 and WO2004/058781. All information disclosed in WO03/55491 and WO2004/058781 which enables the compounds of formula (I) to be prepared, including any necessary protecting group chemistry, is incorporated herein by reference. In particular the methods for preparing 2-{3-[(7-{3-[ethyl(2-hydroxyethyl)amino]propoxy} quinazolin-4-yl)amino]-1H-pyrazol-5-yl}-N-(3-fluorophenyl)acetamide and 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate provided in WO2004/058781 are incorporated herein by reference.

Certain of the efflux transporter inhibitors described herein have been described in: Doyle L A. and Ross D D. (Multidrug Resistance mediated by the Breast Cancer Resistance Protein BCRP (ABCG2), Oncogene 22.47, 2003, 7340-58) and Yanase K. et al. (Functional SNPs of the Breast Cancer Resistance Protein; Therapeutic Effects and Inhibitor Development, Cancer Lett., 2005). They may be prepared by methods known to the skilled person and available in the art, and in particular by the methods referred to in above mentioned literature and the references cited therein.

Other efflux transporter inhibitors described herein have been described in a review by Seelig et al. (Mini-Review in Medicinal Chemistry, 2005, 135-151). They may be prepared by methods known to the skilled person and available in the art, and in particular by the methods referred to in the review by Seeling et al. and the references cited therein. In particular the methods for preparing chlorpromazine, cyclosporin A, diltiazem, nicardipine, progesterone, quinidine, trifluoperazine, verapamil, BIBW22BS, dexniguldipine, gallopamil, PSC833, Roll-2933, trimethoxybenzoylyohimbine, biricodar (VX-710), elacridar (GF120918), zosuquidar (LY335979), MS-209, OC-144-093, laniquidar (R101933), S9788, tariquidar (XR9576), XR9051 and ONT-093 referred to in the review by Seelig et al. are incorporated herein by reference.

It is to be understood that the term “a combination” envisages the simultaneous, sequential or separate administration of the components of the combination. In one aspect of the invention, “a combination” envisages simultaneous administration of a compound of formula (I) and an efflux transporter inhibitor. In a further aspect of the invention, “a combination” envisages sequential administration of those agents. In another aspect of the invention, “a combination” envisages separate administration of those agents. Where the administration of those agents is sequential or separate, the delay in administering the second component should not be such as to lose the benefit of the synergistic effect of the combination therapy. Thus, for the avoidance of doubt, the present invention provides a combination comprising a compound of formula (I), or a pharmaceutically acceptable salt thereof, and an efflux transporter inhibitor for use simultaneously, sequentially or separately in the treatment of hyperproliferative diseases such as cancer.

The treatment of the present invention includes an anti-tumour effect that may be assessed by conventional means such as the response rate, the time to disease progression and/or the survival rate. Anti-tumour effects of the present invention include, but are not limited to, inhibition of tumour growth, tumour growth delay, regression of tumour, shrinkage of tumour, increased time to regrowth of tumour on cessation of treatment and slowing of disease progression. For example, it is expected that when the combination of the present invention is administered to a warm-blooded animal such as a human, in need of treatment for cancer involving a solid tumour, such a method of treatment will produce an effect, as measured by, for example, one or more of: the extent of the anti-tumour effect, the response rate, the time to disease progression and the survival rate.

According to a particular aspect of the present invention there is provided a combination comprising a compound of formula (I) or a pharmaceutically acceptable salt thereof and an efflux transporter inhibitor for use in the treatment of hyperproliferative diseases such as cancer. There is also provided a combination comprising 2-{3-[(7-{3-[ethyl(2-hydroxyethyl)amino]propoxy} quinazolin-4-yl)amino]-1H-pyrazol-5-yl}-N-(3-fluorophenyl)acetamide, 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof and an efflux transporter inhibitor may be selected from resperpine, elacridar, fumitremorgin C (FTC), FTC analogues (such as KO143), BIB-E, flavopiridol, CI1033 (quinazoline), gefitinib (Iressa), novobiocin, biricodar (VX-710), VX-853, diethylstilboestrol (DES), estrone, antioestrogens, tamoxifen and derivatives such as TAG-11, TAG-139, toremifine, imatinib mesylate, CI1033, estradiol, estriol, naringenin, acacetin, kaempferol and naringenin-7-glucoside for use in the treatment of hyperproliferative diseases such as cancer. There is further provided a combination comprising 2-{3-[(7-{3-[ethyl(2-hydroxyethyl)amino]propoxy} quinazolin-4-yl)amino]-1H-pyrazol-5-yl}-N-(3-fluorophenyl)acetamide, 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof and an efflux transporter inhibitor selected from chlorpromazine, cyclosporin A, diltiazem, nicardipine, progesterone, quinidine, trifluoperazine, verapamil, BIBW22BS, dexniguldipine, gallopamil, PSC833, Roll-2933, trimethoxybenzoylyohimbine, biricodar (VX-710), elacridar (GF120918), zosuquidar (LY335979), MS-209, OC-144-093, laniquidar (R101933), S9788, tariquidar (XR9576), XR9051 and ONT-093 for use in the treatment of hyperproliferative diseases such as cancer.

The therapeutic combination of the present invention may be administered in the form of a suitable pharmaceutical composition. According to this aspect of the invention there is provided a pharmaceutical composition for use in the treatment of hyperproliferative diseases such as cancer which comprises a combination as defined hereinbefore in association with a pharmaceutically-acceptable excipient or carrier.

The compositions described herein may be in a form suitable for oral administration, for example as a tablet or capsule, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery. In other embodiments of the present invention the compound of formula (I) of the combination treatment may be delivered endoscopically, intratracheally, intralesionally, percutaneously, intravenously, subcutaneously, intraperitoneally or intratumourally. In general the compositions described herein may be prepared in a conventional manner using conventional excipients or carriers that are well known in the art.

Suitable pharmaceutically acceptable excipients or carriers for a tablet formulation include, for example, inert excipients such as lactose, sodium carbonate, calcium phosphate or calcium carbonate, granulating and disintegrating agents such as corn starch or alginic acid; binding agents such as gelatin or starch; lubricating agents such as magnesium stearate, stearic acid or talc; preservative agents such as ethyl or propyl 4-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

Compositions for oral use may be in the form of hard gelatin capsules in which the active ingredient is mixed with an inert solid excipient, for example, calcium carbonate, calcium phosphate or kaolin, or as soft gelatin capsules in which the active ingredient is mixed with water or an oil such as peanut oil, liquid paraffin or olive oil.

The compositions of the present invention are advantageously presented in unit dosage form.

For further information on formulation the reader is referred to Chapter 25.2 in Volume 5 of Comprehensive Medicinal Chemistry (Corwin Hansch; Chairman of Editorial Board), Pergamon Press 1990.

A compound of formula (I) will generally be administered so that a daily dose in the range, for example, 0.05 mg/kg to 50 mg/kg body weight is received, given if required in divided doses. In general lower doses will be administered when a parenteral route is employed. Thus, for example, for intravenous administration, a dose in the range, for example, 0.05 mg/kg to 25 mg/kg body weight will generally be used. Similarly, for administration by inhalation, a dose in the range, for example, 0.05 mg/kg to 25 mg/kg body weight will be used. Typically, unit dosage forms will contain about 0.05 mg to 25 mg of a compound of formula (I).

An efflux transporter inhibitor may be administered according to known clinical practice.

The dosages and schedules described herein may be varied according to the particular disease state and the overall condition of the patient. For example, it may be necessary or desirable to reduce the above-mentioned doses of the components of the combination treatment in order to reduce toxicity. Dosages and schedules may also vary if, in addition to a combination treatment of the present invention, one or more additional chemotherapeutic agents are used. Scheduling can be determined by the practitioner who is treating any particular patient using his professional skill and knowledge.

It will be appreciated that the pharmaceutical composition according to the present invention includes a composition comprising a compound of formula (I) or a pharmaceutically acceptable salt thereof and an efflux transporter inhibitor and a pharmaceutically-acceptable excipient or carrier. Such a composition conveniently provides the therapeutic combination product of the invention for simultaneous administration in the treatment of hyperproliferative diseases such as cancer.

A pharmaceutical composition according to the present invention also includes separate compositions comprising a first composition comprising a compound of formula (I) or pharmaceutically acceptable salt thereof and a pharmaceutically acceptable excipient or carrier, and a second composition comprising an efflux transporter inhibitor and a pharmaceutically acceptable excipient or carrier. Such a composition conveniently provides the therapeutic combination of the invention for sequential or separate administration in the treatment of cancer or other hyperproliferative disease but the separate compositions may also be administered simultaneously.

Conveniently such a pharmaceutical composition of the invention comprises a kit comprising a first container with a suitable composition containing a compound of formula (I) or a pharmaceutically acceptable salt thereof and a second container with a suitable composition containing an efflux transporter inhibitor. According to this aspect of the present invention there is provided a kit for use in the treatment of hyperproliferative diseases such as cancer comprising:—

a) a compound of formula (I) or pharmaceutically acceptable salt thereof together with a pharmaceutically acceptable excipient or carrier, in a first unit dosage form;
b) an efflux transporter inhibitor together with a pharmaceutically acceptable excipient or carrier, in a second unit dosage form; and
c) container means for containing said first and second dosage forms.

According to this aspect of the invention there is also provided a pharmaceutical composition for use in the treatment of breast, colon, colorectal, lung, prostate, pancreatic or bladder cancer or leukaemia or lymphoma which comprises a combination as defined herein in association with a pharmaceutically acceptable excipient or carrier.

According to a further aspect of the present invention there is provided a combination as defined herein for use in the treatment of hyperproliferative diseases such as cancer.

According to this aspect of the present invention there is also provided a combination as defined hereinbefore for use in the treatment of breast, colon, colorectal, lung, prostate, pancreatic or bladder cancer or leukaemia or lymphoma. The leukaemias and lymphomas mentioned herein maybe tumours of myeloid lineage such as acute myeloid leukaemia or of lymphoid lineage.

According to a further aspect of the present invention there is provided the use of a combination as defined hereinbefore in the manufacture of a medicament for administration to a warm-blooded animal such as man to provide the treatment of hyperproliferative diseases such as cancer.

According to this aspect of the present invention there is also provided the use of a combination as defined hereinbefore in the manufacture of a medicament for administration to a warm-blooded animal such as man to provide the treatment of breast, colon, colorectal, lung, prostate, pancreatic or bladder cancer or leukaemia or lymphoma.

According to a further aspect of the present invention there is provided a method for the treatment of cancer or other hyperproliferative disease which comprises the administration to a warm-blooded animal such as man that is in need of such treatment of effective amounts of the components of the combination as defined herein.

According to this aspect of the present invention there is also provided a method for the treatment of breast, colon, colorectal, lung, prostate, pancreatic or bladder cancer or leukaemia or lymphoma which comprises the administration to a warm-blooded animal such as man that is in need of such treatment of effective amounts of the components of the combination as defined hereinbefore.

According to this aspect of the present invention there is also provided a method for the treatment of hyperproliferative diseases such as cancer which comprises the simultaneous, sequential or separate administration to a warm-blooded animal such as man that is in need of such treatment of effective amounts of the components of the combination as defined hereinbefore.

According to this aspect of the present invention there is also provided a method for the treatment of breast, colon, colorectal, lung, prostate, pancreatic or bladder cancer or leukaemia or lymphoma which comprises the simultaneous, sequential or separate administration to a warm-blooded animal such as man that is in need of such treatment of effective amounts of the components of the combination as defined hereinbefore.

A combination treatment of the present invention as defined hereinbefore may be administered as a sole therapy or may in addition involve surgery or radiotherapy or the administration of an additional chemotherapeutic agent.

Surgery may comprise the step of partial or complete tumour resection, prior to, during or after the administration of the combination treatment of the present invention.

Other chemotherapeutic agents for optional use with the combination treatment of the present invention may include, for example, the following categories of therapeutic agent:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine-threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex vivo and in vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the combination of this invention within the dosage range described hereinbefore and the other pharmaceutically active agent within its approved dosage range.

According to an aspect of the invention there is provided a combination suitable for use in the treatment of cell proliferative disorders (such as cancer) comprising the components of the combination as defined hereinbefore and an additional anti-tumour agent selected from those defined above.

According to this aspect of the invention there is provided a pharmaceutical product comprising the components of the combination as defined hereinbefore and an additional anti-tumour agent as defined above for the conjoint treatment of cancer.

### Preparation of 2-{3-[(7-{3-[ethyl(2-hydroxyethyl)amino]propoxy}-quinazolin-4-yl)amino-]1H-pyrazol-5-yl}-N-(3-fluorophenyl)acetamide

2-(3-{[7-(3-chloropropoxy)quinazolin-4-yl]amino}-1H-pyrazol-5-yl)-N-(3-fluorophenyl)acetamide, potassium iodide, dimethylamine and N-(ethylamino)ethanol were combined and heated to 50° C. for 72 hours. The reaction was diluted with dichloromethane (20 ml) and loaded onto a 40S silica biotage column. Elution with dichloromethane followed by increased polarity to dichloromethane:methanol (9:1), then dichloromethane:methanol:ammonia (9:1:0.8) yielded 2-{3-[(7-{3-[ethyl(2-hydroxyethyl)amino]propoxy}-quinazolin-4-yl)amino]-1H-pyrazol-5-yl}-N-(3-fluorophenyl)acetamide as a yellow solid:

¹H-NMR (DMSO d₆): 12.31 (m, 1H), 10.39 (s, 1H), 10.15 (m, 1H), 8.51 (s, 2H), 7.62 (d, 1H), 7.35 (m, 2H), 7.16 (m, 2H), 6.90 (t, 1H), 6.78 (m, 1H), 4.29 (m, 1H), 4.20 (t, 2H), 3.76 (s, 2H), 3.45 (m, 2H), 3.30 (m, 4H), 2.61 (t, 2H), 1.89 (t, 2H), 0.95 (t, 3H):

MS (+ve ESI): 508.4 (M+H)⁺.

2-(3-{[7-(3-chloropropoxy)quinazolin-4-yl]amino}-1H-pyrazol-5-yl)-N-(3-fluorophenyl)acetamide used as the starting material was made as follows:

a) 2-Amino-4-fluorobenzoic acid was dissolved in 2-methoxyethanol. Formamidine acetate was added and the mixture heated to reflux for 18 hours. The reaction was cooled, concentrated and the residue stirred in aqueous ammonium hydroxide (0.01 N) for 1 hour. The suspension was filtered, washed with water and dried over phosphorus pentoxide to yield 7-fluoroquinazolin-4(3H)-one as an off-white solid:

¹H-NMR (DMSO d₆): 12.32 (br s, 1H), 8.19 (dd, 1H), 8.14 (s, 1H), 7.45 (dd, 1H), 7.39 (m, 1H):

MS (−ve ESI): 163 (M−H)⁻,

MS (+ve ESI): 165 (M+H)⁺.

b) Sodium hydride was added at 0° C. to a solution of 1,3-propanediol in dimethylformamide. 7-Fluoroquinazolin-4(3H)-one was added portionwise and the reaction mixture heated at 60° C., then at 110° C. for 3 hours. The reaction was cooled to 0° C., quenched with water and adjusted to pH 5.9. The resulting suspension was filtered, washed with water then ether and dried over phosphorus pentoxide to afford 7-(3-hydroxypropoxy)quinazolin-4(3H)-one as a white powder:

¹H-NMR (DMSO d₆): 11.90 (br s, 1H), 8.04 (s, 1H), 8.00 (d, 1H), 7.10 (m, 2H), 4.17 (t, 2H), 3.58 (t, 2H), 1.92 (m, 2H):

MS (+ve ESI): 221 (M+H)⁺.

c) 7-(3-hydroxypropoxy)quinazolin-4(3H)-one and thionyl chloride were combined. Dimethylformamide was added and the reaction mixture heated to 85° C. for 1 hour. The mixture was cooled to room temperature, diluted with toluene and evaporated to dryness. This was repeated until all thionyl chloride was removed. The residue was dissolved in dichloromethane and washed with a saturated sodium bicarbonate solution. The aqueous layer was extracted with dichloromethane. The organics were combined, dried (magnesium sulphate) and concentrated to leave a yellow solid. Trituration with ether removed a less soluble impurity and the ether filtrate was concentrated to leave 4-chloro-7-(3-chloropropoxy)quinazoline as an off-white solid (8.5 g, 70% yield):

¹H-NMR (DMSO d₆): 13.25 (br s, 1H), 8.34 (s, 1H), 8.06 (d, 1H), 7.17 (m, 2H), 4.21 (t, 2H), 3.83 (t, 2H), 2.23 (m, 2H):

MS (+ve ESI): 257, 259 (M+H)⁺.

d) 4-chloro-7-(3-chloropropoxy)quinazoline and (3-amino-1H-pyrazol-5-yl)acetic acid were combined in dimethylformamide. A solution of 4M HCl in dioxane was added and the reaction heated to 90° C. for 40 minutes. The solution was cooled to room temperature, diluted with water and filtered through celite. The acidic solution was basified to pH 4.9 and the yellow powder filtered. (At pH 3, a red solid precipitated which was isolated, suspended in water and basified to pH 12. Careful adjustment back to pH 4.8 resulted in the precipitation of a yellow powder, which was combined with the first crop). The solid was washed with diethyl ether and dried over phosphorus pentoxide to yield (3-{[7-(3-chloropropoxy)quinazolin-4-yl]amino}-1H-pyrazol-5-yl)acetic acid as a pale orange solid:

¹H-NMR (DMSO d₆): 12.60 (br s, 2H), 10.78 (br s, 1H), 8.65 (s, 1H), 8.60 (d, 1H), 7.26 (d, 1H), 7.22 (s, 1H), 6.67 (s, 1H), 4.28 (t, 2H), 3.83 (t, 2H), 3.67 (s, 2H), 2.24 (m, 2H):

MS (−ve ESI): 360, 362 (M−H)⁻,

MS (+ve ESI): 362, 364 (M+H)⁺.

e) 3-Fluoroaniline was added to a suspension of (3-{[7-(3-chloropropoxy)quinazolin-4-yl]amino}-1H-pyrazol-5-yl)acetic acid in pyridine and the reaction cooled to 0° C. Phosphorous oxychloride was added dropwise and the reaction stirred at 0° C. for 1 hour. The reaction was warmed to ambient temperature and more phosphorous oxychloride added. The reaction was stirred for 4.5 hours. The reaction mixture was diluted with ethyl acetate: ether (100 ml: 37 ml) and stirred for 18 hours. The precipitate was filtered, suspended in water and neutralised with ammonium hydroxide (7%). The resultant yellow suspension was filtered, washed with water and dried (phosphorous pentoxide) to yield 2-(3-{[7-(3-chloropropoxy)quinazolin-4-yl]amino}-1H-pyrazol-5-yl)-N-(3-fluorophenyl)acetamide as an orange powder.

### Preparation of 2-{ethyl[3-({4-[(5-[2-[(3-fluorophenyl)amino]-2-oxoethyl]-1H-pyrazol-3-yl)amino]quinazolin-7-yl}oxy)propyl]amino}ethyl dihydrogen phosphate

Di(tert-butyl) 2-[[3-({4-[(5-{2-[(3-fluorophenyl)amino]-2-oxoethyl}-1H-pyrazol-3-yl)amino]-quinazolin-7-yl}oxy)propyl](ethyl)amino]ethyl phosphate was suspended in dioxane and treated with a solution of hydrochloric acid (4.0 N) in dioxane at ambient temperature for 15 hours. The solid was recovered by filtration, washed with dioxane, dried in vacuo at 50° C. to yield the title compound as a pale yellow dihydrochloride salt:

¹H-NMR (DMSO d₆): 11.98 (s, 1H), 10.79 (s, 1H), 8.93 (s, 1H), 8.83 (d, 1H), 7.65 (d, 1H), 7.47 (d, 1H), 7.38 (m, 3H), 6.89 (t, 1H), 6.74 (s, 1H), 4.32 (t, 2H), 4.28 (m, 2H), 3.85 (s, 2H), 3.42 (m, 2H), 3.34 (m, 2H), 3.27 (q, 2H), 2.29 (m, 2H), 1.28 (t, 3H):

MS (+ve ESI): 587.8 (M+H)⁺.

Di(tert-butyl) 2-[[3-({4-[(5-{2-[(3-fluorophenyl)amino]-2-oxoethyl}-1H-pyrazol-3-yl)amino]-quinazolin-7-yl}oxy)propyl](ethyl)amino]ethyl phosphate used as the starting material was prepared as follows:

Di-tert-butyl-diethylphosphoramidite (417 μm, 1.5 mmol) was slowly added to a solution of 2-{3-[(7-{3-[ethyl(2-hydroxyethyl)amino]propoxy} quinazolin-4-yl)amino]-1H-pyrazol-5-yl}-N-(3-fluorophenyl)acetamide in dimethylformamide in the presence of tetrazole at ambient temperature under argon. The mixture was stirred at ambient temperature for 1.5 hours, cooled to −10° C. and hydrogen peroxide (9.0 N solution) was slowly added. The resulting mixture was stirred at ambient temperature for 2 hours. Sodium metabisulphite in water was then added at 0° C. and the mixture was stirred at ambient temperature for 0.5 hour. The mixture was concentrated, dichloromethane/methanol (8:2) was added before the solid was filtered and washed with dichloromethane/methanol. Concentration of the filtrate in vacuo followed by chromatography on silica gel, eluting with dichloromethane/methanol (90:10) to dichloromethane/methanol/ammonia (7.0 N) (90:10:1), yielded di-tert-butyl 2-[[3-({4-[(5-{2-[(3-fluorophenyl)amino]-2-oxoethyl}-1H-pyrazol-3-yl)amino]quinazolin-7-yl}oxy)propyl](ethyl)amino]ethyl phosphate as a pale yellow solid.

2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate, synthesised above as the dihydrochloride salt, was also prepared as the free base according to the following method:

2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate, synthesised above as the dihydrochloride salt was solubilized in methanol and cyclohexane oxide was added to the solution. The solution was stirred at ambient temperature for 48 hours, during which time a white solid precipitated. The mixture was diluted with diethyl ether (100 ml) and the solid was recovered by filtration, washed with ether and dried in vacuo to give the free base of 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate as a pale yellow solid:

¹H-NMR (DMSO d₆): 10.53 (s, 1H), 8.57 (s, 1H), 8.54 (d, 1H), 7.62 (d, 1H), 7.37 (m, 2H), 7.27 (s, 1H), 7.21 (d, 1H), 6.88 (m, 1H), 6.65 (s, 1H), 4.27 (t, 2H), 4.05 (m, 2H), 3.75 (s, 2H), 3.24 (m, 2H), 3.21 (t, 2H), 3.13 (q, 2H), 2.18 (m, 2H), 1.24 (t, 3H):

MS (+ve ESI): 588 (M+H)⁺.

C₂₆H₃₁FN₇O₆P+3.0H₂O requires C, 48.7%; H, 5.8%; N, 15.3%; Found C, 48.8%; H, 5.35%; N, 15.15%.

### Preparation of 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate (Alternative Route(s))

### Preparation of 7-(3-hydroxypropoxy)quinazolin-4(3H)-one

2-Amino-4-fluorobenzoic acid and 1,3-propanediol were stirred together and heated to 120° C. Formamidine acetate was added and the mixture stirred for 3.5 hour to yield 7-fluoroquinazoline-4-one. A solution of potassium hydroxide in 1,3-propanediol was then added to the mixture over a period of 2 hours and 50 minutes, which was then cooled 15° C. Following this, the mixture was heated to 125° C. for 5 hour before cooling to 75° C. Dilute hydrochloric acid (about 6% w/w) was gradually added to the reaction mixture until pH 4.5 was achieved. The mixture was cooled to 0° C. over 6 hour and maintained at that temperature for a further hour prior to isolation of the crude product by centrifugation. The crude material was washed with water and dried in vacuo before dissolving in methanol at gentle reflux and partially concentrating under reduced pressure at a temperature of 42° C. This solution was then cooled to 0° C. over a period of 3 hour and the resultant product was isolated by filtration, prior to drying in vacuo. 7-(3-Hydroxypropoxy)quinazolin-4(3H)-one was recovered in a 73% yield.

¹H-NMR (DMSO d₆): 11.90 (br s, 1H), 8.04 (s, 1H), 8.00 (d, 1H), 7.10 (m, 2H), 4.17 (t, 2H), 3.58 (t, 2H), 1.92 (m, 2H)

MS (+ve ESI): 221 (M+H)⁺

### Preparation of 4-chloro-7-(3-chloropropoxy)quinazoline

7-(3-Hydroxypropoxy)quinazolin-4(3H)-one, toluene and N,N-diisopropyl-formamide (DIPF) were mixed together and heated to 76° C., before thionyl chloride was added over a period of 1 hour at 76° C. Additional thionyl chloride was then added over a period of 1 hour after which the temperature was maintained at 76° C. for 1 hour. The mixture was refluxed for 11 hours to effect a clear solution which was cooled to 38° C. and subjected to vacuum distillation to remove toluene and thionyl chloride. Toluene was then added and the solution kept at 35° C. whilst it was clarified with a filter aid (celite or harborlite and activated carbon). The resulting solution was partially concentrated before heptane was added and the mixture chilled to 0° C. and stirred for 23 hours. The light brown suspension that formed was isolated by filtration, washed with cold heptane then dried in vacuo at 30° C. to yield 4-chloro-7-(3-chloropropoxy)quinazoline (63.6%)

¹H-NMR (DMSO d₆): 13.25 (br s, 1H), 8.34 (s, 1H), 8.06 (d, 1H), 7.17 (m, 2H), 4.21 (t, 2H), 3.83 (t, 2H), 2.23 (m, 2H):

MS (+ve ESI): 257, 259 (M+H)⁺.

### Preparation of (3-{[7-(3-chloropropoxy)quinazolin-4-yl]amino}-1H-pyrazol-5-yl)acetic acid

4-Chloro-7-(3-chloropropoxy)quinazoline was added to 1 molar equivalent of a solution of (3-amino-1H-pyrazol-5-yl)acetic acid in N-methylpyrrolidinone (NMP) and then left for a period of 12 hours. Crystallisation of the product was observed to occur with and without seeding and with and without the addition of acetonitrile as an anti-solvent. The resultant solid was isolated by filtration, washed with N-methylpyrrolidinone and acetonitrile and then dried in vacuo to yield (3-{[7-(3-chloropropoxy)quinazolin-4-yl]amino}-1H-pyrazol-5-yl)acetic acid hydrochloride as an off-white solid containing one molar equivalent of NMP:

¹H-NMR (DMSO d₆): 8.92 (s, 1H), 8.79 (d, 1H), 7.45 (pr of d, 1H), 7.38 (d, 1H), 6.7 (s, 1H), 6.67 (s, 1H), 4.31 (t, 2H), 3.85 (t, 2H), 3.72 (s, 2H), 3.3 (t), 2.7 (s,), 2.27 (m, 2H), 2.18 (t), 1.9 (m):

MS (+ve ESI): 362.1015 (M+H)⁺.

### Preparation of 2-(3-{[7-(3-chloropropoxy)quinazolin-4-yl]amino}-1H-pyrazol-5-yl)-N-(3-fluorophenyl)acetamide

To a suspension of (3-{[7-(3-chloropropoxy)quinazolin-4-yl]amino}-1H-pyrazol-5-yl)acetic acid hydrochloride in N,N-dimethylacetamide (DMA) is added 4-dimethylaminopyridine (DMAP) whilst maintaining a temperature of 15-25° C. (ideally 15° C.) followed by N-methylmorpholine whilst also maintaining the temperature. 3-Fluoroaniline (in a large excess which ideally is between 10-15 mole equivalents) is added at such a rate as to maintain the temperature below 25° C. Meanwhile 1-ethyl-3-(3-dimethylaminopropyl)carbodiimide hydrochloride (EDCI.HCl) is dissolved in water to afford a solution about 42% w/v (the quantity of water present is important to the outcome of the crystallisation later in the process). This solution is added in a controlled manner to the slurry over a period of 8 hour so as to maintain the reaction between 20-25° C.; then the mixture is seeded with crystals of the preferred form of the product (ideally an amount of about 1% of the expected yield). The mixture is stirred for about 16 hours whilst maintaining the temperature (ideally 20-25° C.) then anti-solvents acetonitrile followed by water are added in a controlled manner and to maintain the temperature between 20-25° C. followed by an extended stir of about 21 hours; this is to optimise the recovery and form of the product. The material is isolated by filtration and the cake washed with a mixture of N,N-dimethylacetamide: water:acetonitrile (volume ratios of 5:3:2), acetonitrile and then dried (in vacuo or under a stream of nitrogen) to afford 2-(3-{[7-(3-chloropropoxy)quinazolin-4-yl]amino}-1H-pyrazol-5-yl)-N-(3-fluorophenyl)acetamide containing some DMA in about 76-78% yield.

¹H-NMR (DMSO d₆; contains residual DMA): 10.4 (s, 1H), 8.9 (s, 1H), 8.8 (d, 1H), 7.59 (pr of m, 1H), 7.46 (pr of d, 1H), 7.33 (m, 2H), 7.29 (d, 1H), 6.85 (m, 1H), 6.75 (s, 1H), 4.35 (t, 2H), 3.85 (t, 4H), 2.95 (s), 2.83 (s), 2.56 (s), 2.25 (m, 2H), 1.95 (s):

MS (+ve): 455 (M+H)⁺.

### Preparation of 2-{3-[(7-{3-[ethyl(2-hydroxyethyl)amino]propoxy}-quinazolin-4-yl)amino]-1H-pyrazol-5-yl}-N-(3-fluorophenyl acetamide

2-(3-{[7-(3-Chloropropoxy)quinazolin-4-yl]amino}-1H-pyrazol-5-yl)-N-(3-fluorophenyl)acetamide and 2-(ethylamino)ethanol (ideally 12 molar equivalents) were added to N,N-dimethylacetamide under an inert atmosphere (such as provided by nitrogen) and the mixture heated to 90° C. with stirring. After a period of 12-16 hours (ideally 12 hours) the reaction is cooled back to about 85° C. and water added in a controlled manner to maintain the temperature between 80-85° C. The batch is adjusted to 80° C. and seeded with crystals of the preferred form of the product (ideally an amount of about 1% of the expected yield). The mixture was cooled to 20° C. in a carefully controlled manner over a period of about 20 hours so as to crystallise the product in the required form and of a size sufficient to afford a good filtration rate. The product is then filtered and washed with a mixture of water/N,N-dimethylacetamide and acetonitrile and suitably deliquored to afford a hydrated form of the product. Following this, the cake is slurried in situ for a period (ideally 2 hours) with warm acetonitrile (ideally at a temperature of 40° C.) then filtered, washed with more acetonitrile and then dried (in vacuo or under a stream of nitrogen) to afford the almost anhydrous 2-{3-[(7-{3-[ethyl(2-hydroxyethyl)amino]propoxy}-quinazolin-4-yl)amino]-1H-pyrazol-5-yl}-N-(3-fluorophenyl)acetamide as an off-white solid in a yield of 85-90%.

¹H-NMR (DMSO d₆): 10.55 (s, 1H), 9.45 (br s, 1H), 8.98 (s, 1H), 8.8 (d, 1H), 7.63 (pr of m, 1H), 7.47 (pr of d, 1H), 7.37 (m, 2H), 7.32 (d, 1H), 6.9 (m, 1H), 6.77 (s, 1H), 4.32 (t, 2H), 3.83 (br s, 2H), 3.76 (t, 2H), 3.35 (m, 2H), 3.25 (m, 4H), 2.25 (m, 2H), 1.25 (t, 3H):

MS (+ve ESI): 508.4 (M+H)⁺.

### Preparation of mono(tert-butyl) 2-[[3-({4-[(5-{2-[(3-fluorophenyl)amino]-2-oxoethyl}-1H-pyrazol-3-yl)amino]-quinazolin-7-yl}oxy)propyl](ethyl)amino]ethyl phosphate

2-{3-[(7-{3-[Ethyl(2-hydroxyethyl)amino]propoxy}-quinazolin-4-yl)amino]-1H-pyrazol-5-yl}-N-(3-fluorophenyl)acetamide and pyridine.hydrochloride were mixed in N,N-dimethylacetamide and the solution chilled to −15° C. Di-tert-butyl diethylphosphoramidite (1.5-2.1 molar equivalents) was then added whilst the temperature was maintained. The reaction mixture was treated in situ with 30% w/w hydrogen peroxide (about 4.2 mole equivalents) whilst the temperature was kept below ambient temperature. Remaining hydrogen peroxide was destroyed by the addition of sodium metabisulphite (as a 10% w/v solution) whilst maintaining the temperature below 40° C. The resulting solution of di-tert-butyl 2-[[3-({4-[(5-{2-[(3-fluorophenyl)amino]-2-oxoethyl}-1H-pyrazol-3-yl)amino]quinazolin-7-yl}oxy)propyl](ethyl)amino]ethyl phosphate was then heated to 40° C. and sodium hydroxide solution (2M) added to adjust to pH 5-6.5. The temperature and pH was maintained for a period of about 90 minutes with seeding. Water was then charged and the pH adjusted further to within the range pH 8-9 to optimise the recovery. The warm reaction mixture was filtered directly to afford mono-tert-butyl 2-[[3-({4-[(5-{2-[(3-fluorophenyl)amino]-2-oxoethyl}-1H-pyrazol-3-yl)amino]quinazolin-7-yl}oxy)propyl](ethyl)amino]ethyl phosphate which was washed with a mixture of N,N-dimethylacetamide/water and water and finally dried (in vacuo or a stream of a suitable inert gas) to afford mono(tert-butyl) 2-[[3-({4-[(5-{2-[(3-fluorophenyl)amino]-2-oxoethyl}-1H-pyrazol-3-yl)amino]-quinazolin-7-yl}oxy)propyl](ethyl)amino]ethyl phosphate as an off-white solid at a yield of between 86-93%.

¹H-NMR (DMSO d₆): 10.48 (s, 1H), 9.75 (br s, 1H), 8.98 (s, 1H), 8.85 (d, 1H), 7.67 (pr of m, 1H), 7.48 (pr of d, 1H), 7.37 (m, 2H), 7.3 (d, 1H), 6.87 (m, 1H), 6.83 (s, 1H), 4.34 (t, 2H), 4.28 (m, 2H), 3.88 (s, 2H), 3.53 (m, 2H), 3.43 (m, 2H), 3.33 (m, 2H), 2.3 (m, 2H), 1.47 (s, 9H), 1.32 (t, 3H):

MS (+ve ESI): (M+H)⁺ 644.2761 fragment (less butyl) 588.2147

### Preparation of mono(tert-butyl) 2-{[3-([4-[(5-{2-[(3-fluorophenyl)amino]-2-oxoethyl}-1H-pyrazol-3-yl)amino]-quinazolin-7-yl}oxy)propyl](ethyl)amino]ethyl phosphate

### Alternative Route

To a slurry of pyridine hydrochloride in N,N-dimethylacetamide was charged a solution of 2-{3-[(7-{3-[Ethyl(2-hydroxyethyl)amino]propoxy}-quinazolin-4-yl)amino]-1H-pyrazol-5-yl}-N-(3-fluorophenyl)acetamide and di-tert-butyl diethylphosphoramidite (ideally 1 molar equivalents) in N,N-dimethylacetamide over an extended period (ideally 3 hours) and maintaining the temperature between −20 to −10° C. (ideally −15° C.). This is followed by the further addition of di-tert-butyl diethylphosphoramidite (ideally 0.5 molar equivalents) during a period of 1 hour also maintaining the temperature between −20 to −10° C. (ideally −15° C.).

The reaction mixture is treated in situ with 30% w/w hydrogen peroxide (about 4.2 mole equivalents) whilst the temperature was kept below −10° C. (ideally −12 to −8° C.) and held for a period at this temperature (ideally 16 hours). Remaining hydrogen peroxide is destroyed by the addition of sodium metabisulphite (as a 10% w/v aqueous solution) whilst maintaining the temperature below 40° C.

The resulting solution of di-tert-butyl 2-[[3-({4-[(5-{2-[(3-fluorophenyl)amino]-2-oxoethyl}-1H-pyrazol-3-yl)amino]quinazolin-7-yl}oxy)propyl](ethyl)amino]ethyl phosphate was then heated to 40° C. and sodium hydroxide solution (ideally 2M) added to adjust to pH 5.5-6.5 (ideally pH 6) with seeding with suitably crystalline material. The temperature is held and a range of pH 5-6 maintained by the addition of extra sodium hydroxide solution for a period of at least 2 hours. Water is then charged and the pH adjusted further to within the range pH 8-9 (ideally pH 8.8) whilst maintaining the temperature (ideally 40° C. but within range 35-45° C.) for a period of 16 hours so as to optimise the recovery. The warm reaction mixture is filtered directly to afford mono-tert-butyl 2-[[3-({4-[(5-{2-[(3-fluorophenyl)amino]-2-oxoethyl}-1H-pyrazol-3-yl)amino]quinazolin-7-yl}oxy)propyl](ethyl)amino]ethyl phosphate which was washed several times with water and finally dried (either in vacuo or a stream of a suitable inert gas) to afford the mono(tert-butyl) 2-[[3-({4-[(5-{2-[(3-fluorophenyl)amino]-2-oxoethyl}-1H-pyrazol-3-yl)amino]-quinazolin-7-yl}oxy)propyl](ethyl)amino]ethyl phosphate as an off-white solid at a yield of between 86-93%.

¹H-NMR (DMSO d₆): 10.48 (s, 1H), 9.75 (br s, 1H), 8.98 (s, 1H), 8.85 (d, 1H), 7.67 (pr of m, 1H), 7.48 (pr of d, 1H), 7.37 (m, 2H), 7.3 (d, 1H), 6.87 (m, 1H), 6.83 (s, 1H), 4.34 (t, 2H), 4.28 (m, 2H), 3.88 (s, 2H), 3.53 (m, 2H), 3.43 (m, 2H), 3.33 (m, 2H), 2.3 (m, 2H), 1.47 (s, 9H), 1.32 (t, 3H):

MS (+ve ESI): (M+H)⁺ 644.2761 fragment (less butyl) 588.2147.

### Preparation of 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate

Mono(tert-butyl) 2-[[3-({4-[(5-{2-[(3-fluorophenyl)amino]-2-oxoethyl}-1H-pyrazol-3-yl)amino]-quinazolin-7-yl}oxy)propyl](ethyl)amino]ethyl phosphate was suspended in a mixture of water/tetrahydrofuran (THF) and treated with an excess of between 1.5 and 3.0 molar equivalents of hydrochloric acid (ideally of a concentration of 2M and containing 1.5 mole equivalents). The mixture is heated to 55-65° C. (ideally 60° C.) and held at 60° C. for about 1 hour. The hot solution is then basified using sodium hydroxide (preferably of 2M concentration and containing 1.7 mole equivalents) to afford a pH within the range pH 5.0-5.5 and then seeded at 55-65° C. (ideally 60° C.) with crystals of the preferred form of the product (ideally an amount of about 0.05% w/w of the expected yield). The mixture is stirred at this temperature for at least one hour before water is added and the slurry stirred and cooled in a controlled manner over a period of about 12 hours prior to stirning at ambient temperature for at least 4 hours and then isolating the product by filtration. The filter-cake is washed successively with water then THF and dried either in vacuo or using a humidification procedure whereby an inert gas dampened with water vapour is passed over the solid until a constant weight is obtained. After the drying in vacuo the solid 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate is equilibrated under ambient conditions to constant weight to give a hydrated form as a pale yellow needle-like material. The product is obtained in about 81% yield.

¹H-NMR (DMSO d₆):

MS (+ve ESI): 587.8 (M+H)⁺

¹H-NMR (DMSO d₆): 10.53 (s, 1H), 8.57 (s, 1H), 8.54 (d, 1H), 7.62 (d, 1H), 7.37 (m, 2H), 7.27 (s, 1H), 7.21 (d, 1H), 6.88 (m, 1H), 6.65 (s, 1H), 4.27 (t, 2H), 4.05 (m, 2H), 3.75 (s, 2H), 3.24 (m, 2H), 3.21 (t, 2H), 3.13 (q, 2H), 2.18 (m, 2H), 1.24 (t, 3H):

MS (+ve ESI): 588 (M+H)⁺.

C₂₆H₃₁FN₇O₆P+3.0H₂O requires C, 48.7%; H, 5.8%; N, 15.3%; Found C, 48.8%; H, 5.35%; N, 15.15%.

### Activity in MDR Cells

The following test method may be used to demonstrate the activity of 2-[[3-({4-[(5-{2-[(3-fluorophenyl)amino]-2-oxoethyl}-1H-pyrazol-3-yl)amino]-quinazolin-7-yl}oxy)propyl](ethyl)amino]ethyl dihydrogen phosphate in MDR cells.

### Method

The UIC2 monoclonal antibody (Immunotech mouse monoclonal IgG2a antibody clone UIC2 cat no: 1864) reacts specifically with an extracellular epitope of Pgp and inhibits the efflux of transported drugs in MDR cells. mAb UIC2 was used to specifically inhibit Pgp mediated efflux and to increase the activity of 2-[[3-({4-[(5-{2-[(3-fluorophenyl)amino]-2-oxoethyl}-1H-pyrazol-3-yl)amino]-quinazolin-7-yl}oxy)propyl](ethyl)amino]ethyl dihydrogen phosphate in MDR cells.

MCF7 ADR cells are known to highly express Pgp and other efflux transporters.

On day 1; 8×10⁴ MCF7 ADR cells/ml were plated into a 96 well plate in 100 μl of media (DMEM, 10% FCS, 1% glutamine) and left to adhere at 37° C. overnight.

On day 2; 3 wells of MCF7 ADR cells were left untreated; 3 wells of MCF7 ADR cells were treated with 100 nM of aurora kinase inhibitor; 3×3 wells of MCF7 ADR cells were incubated with different concentrations of UIC2 mAb at 37° C. for 30 minutes (10 μg/ml, 1 μg/ml, and 0.1 μg/ml based on the recommended working concentrations provided by the supplier) and then dosed with 100 nM of aurora kinase inhibitor (a dose known to be inactive); and 3 wells of MCF7 ADR cells were incubated with the mouse IgG1 (Dako X0931) at 37° C. for 30 minutes and then dosed with 100 nM of aurora kinase inhibitor.

After 24 hours the cells were examined using the light microscope, and any cellular change in morphology was noted. The cells were then fixed with 100 μl of 3.7% formaldehyde for 30 minutes at room temperature and then washed in PBS using an automated plate washer. 1001 PBS 0.5% triton X-100 was then added for 5 minutes on a shaker. The plate was washed in 100 μl PBS and 50 μl of primary antibody (1:1000 rabbit anti-phosphohistone H3 in PBS 1% BSA 0.5% tween) was added. The plate was left for at least 1 hour at room temperature, on a shaker, after which the antibody was removed and the cells washed twice with PBS. In darkness, 50 μl of secondary antibody (1:10,000 Hoechst and 1:1000 Alexa Fluor 488 anti rabbit in PBS 1% BSA 0.5% tween) was added and the plate left for at least 1 hour at room temperature. The secondary antibody was removed and the plate washed twice with PBS. 200 μl PBS was then added and left for 10 minutes with shaking. The PBS was removed. 100 μl PBS was added and the plate sealed prior to analysis. (The plate was stored in the dark at 4° C. prior to analysis).

The plate was analysed on a Cellomics Arrayscan using the Target Activation algorithm. The primary endpoint was the inhibition of phosphohistone H3 (phH3), which is a biomarker, associated with aurora B inhibition.

Results

A large activity is associated with an increased inhibition of the biomarker phH3. It is thus clear that UIC2 mAb (10 μg/ml) inhibited the Pgp mediated efflux of 2-[[3-({4-[(5-{2-[(3-fluorophenyl)amino]-2-oxoethyl}-1H-pyrazol-3-yl)amino]-quinazolin⁻⁷-yl}oxy)propyl](ethyl)amino]ethyl dihydrogen phosphate.

The activity of 2-[[3-({4-[(5-{2-[(3-fluorophenyl)amino]-2-oxoethyl}-1H-pyrazol-3-yl)amino]-quinazolin-7-yl}oxy)propyl](ethyl)amino]ethyl dihydrogen phosphate in cells that are known to express BRCP may also be demonstrated using an analogous assay to that described herein but replacing MCF7 ADR cells with A529 cells, a human lung carcinoma cell line known in the art and by using a BCRP specific antibody (Chemicon mouse monoclonal antibody BXP-21 cat. no. 1864).

In addition to the use of antibodies, it would also be possible to inhibit BRCP or Pgp function using small molecule inhibitors, such as those described herein using an analagous procedure to that described herein but replacing the antibodies with the small molecule inhibitors.

1. A combination comprising an aurora kinase inhibitor and an efflux transporter inhibitor wherein the aurora kinase inhibitor is a compound of formula (I) or pharmaceutically acceptable salt thereof: 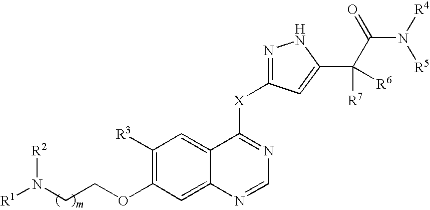
wherein:
m is 0, 1, 2 or 3
R¹ is hydroxyC₁₋₄alkyl or phosphonooxyC₁₋₄alkyl;
R² is hydrogen, C₁₋₄alkyl, hydroxyC₁₋₄alkyl, C₁₋₄alkoxyC₁₋₄alkyl or heterocyclyl;
or R¹ and R² together with the nitrogen to which they are attached form a 4- to 6-membered heterocyclic ring optionally containing a further heteroatom selected from nitrogen, oxygen and sulphur which nitrogen or sulphur is optionally oxidised and which ring is optionally substituted with C₁₋₄alkyl;
R³ is hydrogen or C₁₋₄alkoxy;
R⁴ is hydrogen or C₁₋₄alkyl;
R⁵ is aryl optionally substituted by 1 or 2 halo; and
R⁶ and R⁷ are independently hydrogen or C₁₋₄alkyl.

2. A combination according to claim 1 wherein the aurora kinase inhibitor is 2-{3-[(7-{3-[ethyl(2-hydroxyethyl)amino]propoxy}quinazolin-4-yl)amino]-1H-pyrazol-5-yl}-N-(3-fluorophenyl)acetamide, 2-{ethyl[3-({4-[(5-{2-[(3-fluorophenyl)amino]-2-oxoethyl}-1H-pyrazol-3-yl)amino]quinazolin-7-yl}oxy)propyl]amino}ethyl dihydrogen phosphate or a pharmaceutically acceptable salt thereof.

3. A combination according to claim 2 wherein the efflux transporter inhibitor is selected from resperpine, elacridar, fumitremorgin C (FTC), FTC analogues (such as KO143), BIB-E, flavopiridol, CI1033 (quinazoline), gefitinib (Iressa), novobiocin, biricodar (VX-710), VX-853, diethylstilboestrol (DES), estrone, antioestrogens, tamoxifen and derivatives such as TAG-11, TAG-139, toremifine, imatinib mesylate, CI1033, estradiol, estriol, naringenin, acacetin, kaempferol and naringenin-7-glucoside.

4. A combination according to claim 2 wherein the efflux transporter inhibitor is selected from chlorpromazine, cyclosporin A, diltiazem, nicardipine, progesterone, quinidine, trifluoperazine, verapamil, BIBW22BS, dexniguldipine, gallopamil, PSC833, Roll-2933, trimethoxybenzoylyohimbine, biricodar (VX-710), elacridar (GF120918), zosuquidar (LY335979), MS-209, OC-144-093, laniquidar (R101933), S9788, tariquidar (XR9576), XR9051 and ONT-093.

5. (canceled)

6. A pharmaceutical composition for use in the treatment of hyperproliferative diseases such as cancer which comprises a combination as defined in claim 1 in association with a pharmaceutically-acceptable excipient or carrier.

7. A pharmaceutical composition comprising a first composition comprising a compound of formula (I) or pharmaceutically acceptable salt thereof as defined in claim 1 and a pharmaceutically-acceptable excipient or carrier, and a second composition comprising an efflux transporter inhibitor and a pharmaceutically-acceptable excipient or carrier.

8. A kit for use in the treatment of hyperproliferative diseases such as cancer comprising:—
a) a compound of formula (I) or pharmaceutically acceptable salt thereof as defined in claim 1 together with a pharmaceutically acceptable excipient or carrier, in a first unit dosage form;
b) an efflux transporter inhibitor together with a pharmaceutically acceptable excipient or carrier, in a second unit dosage form; and
c) container means for containing said first and second dosage forms.

9. (canceled)

10. A method for the treatment of cancer or other hyperproliferative disease which comprises the administration to a warm-blooded animal such as man that is in need of such treatment of effective amounts of the combination as defined in claim 1.

11. A method for the treatment of hyperproliferative diseases such as cancer which comprises the simultaneous, sequential or separate administration to a warm-blooded animal such as man that is in need of such treatment of effective amounts of the combination as defined in claim 1.
